(19) United States
(12) Patent Application Publication
(10) Pub. No.: US 2019/0027246 A1
(43) Pub. Date: Jan. 24, 2019

(54) METHOD AND APPARATUS FOR DETECTING AND ANALYZING A WORKFLOW PERFORMED WITH AN IMAGING APPARATUS

(71) Applicant: Siemens Healthcare GmbH, Erlangen (DE)

(72) Inventors: George William Ferguson, Erlangen (DE); Stephan Nufer, Erlangen (DE)

(73) Assignee: Siemens Healthcare GmbH, Erlangen (DE)

(21) Appl. No.: 16/042,467

(22) Filed: Jul. 23, 2018

(30) Foreign Application Priority Data
Jul. 24, 2017 (EP) 17182771

(51) Int. Cl. G16H 40/20; G06Q 10/06; G06T 7/292; G06K 9/00

(52) U.S. Cl. CPC G16H 40/20; G06Q 10/0633; G06K 9/00771; G06K 9/00342; G06T 7/292

(57) ABSTRACT

In a method and imaging apparatus for detecting and analyzing a workflow performed in an examination room of the imaging apparatus during a patient examination, first detection data of a first position on or close to a patient table in the examination room, and recording second detection data of a second position anywhere else in the examination room, are recovered by a detection unit, continuously over time. The detection data are analyzed in order to identify whether at least one action has taken place in the examination room, and record the time and/or the time interval in which the at least one action has taken place, and identify the type of the at least one action that has taken place, and recording the type of the at least one action.

### BACKGROUND OF THE INVENTION

### Field of the Invention

The present invention relates to a method for detecting and analyzing a workflow performed with an imaging apparatus. Thus, the present invention relates to the technical field of performing an examination of a patient with an imaging apparatus, for example, a computer tomography apparatus, a magnetic resonance apparatus, or the like.

### Description of the Prior Art

Currently, to gather statistics about a duration of an examination, single work steps performed during an examination are recorded manually using a stopwatch. However, recording single work steps during an examination is time-consuming and inaccurate. As a result, the analysis of the workflow composed of work steps performed is inexact, since only the start and the end of an acquisition can be detected accurately. Moreover, no exact data can be acquired about the time intervals in which an imaging apparatus is not used.

Therefore, the usage of an imaging apparatus is usually not optimized.

For example, DE 10 2015 211 148 A1 discloses the detection of positioning data of a patient when the patient is placed on a patient table by a positioning detection unit. The position data detected reproduce a surface image of the patient, i.e., the anatomy of the patient.

In order to optimize the usage of an imaging apparatus, however, information about the image acquisition time needed for performing an examination of a patient and information about the time or time intervals for performing work steps in preparation and/or follow-up work steps are needed.

### SUMMARY OF THE INVENTION

An object of the present invention is to provide a method for improving a utilization rate of an imaging apparatus and to provide an imaging apparatus that can be used effectively.

According to the invention, the method for detecting and analyzing a workflow consisting of work steps performed in an examination room of an imaging apparatus, in particular, during a patient examination has the following steps.

First detection data of a first position on or close to a patient table in the examination room are recorded and/or second detection data of at least a second position anywhere else in the examination room are recorded, by at least one detection unit. The first and second detection data are continuously recorded as a function of time.

The first and/or second detection data are analyzed by an analysis processor, by identifying whether at least one action has taken place in the examination room, recording the time and/or the time interval in which the at least one action has taken place, and identifying the type of the at least one action which has taken place, and recording the type of the at least one action.

The analysis processor thereby creates an archive record of the detected actions as they occur during the examination, and this archive record is made available from the analysis processor for review and further evaluation of the examination.

In the method according to the invention, the work steps of preparing an examination, performing the same, and processing follow-up work steps are recorded by the detection unit. Since the different work steps are recorded continuously as a function of time, the different work steps are recorded accurately. This allows a detailed analysis of the workflow of an imaging apparatus, for example, a computer tomography apparatus, a magnetic resonance apparatus, or the like. Since a first position is detected, which indicates whether an action has taken place in the area of the patient table, and since at least a second position anywhere else in the examination room is detected which indicates whether an action has taken place in another area of the examination room, the detected data can be recorded accurately and completely, allowing an reliable analysis of the usage of the imaging apparatus, i.e., the utilization rate of the imaging apparatus. The actions in the other area can be, for example, detecting whether a coil needed for the examination is placed on the subject, monitoring access to the examination room, or the like.

According to an embodiment of the invention, the step of identifying whether at least one action has taken place in the examination room is based on a movement detection. The movement detection could be performed using a camera and/or a movement sensor. The movement detection allows for continuous and accurate detection of whether an action has taken place, e.g. whether a patient or an operator has moved or has been moved in some way, and detection of how much time was needed for performing this action.

According to a further embodiment of the invention, the step of identifying the type of the at least one action is based on a detection data analysis of the detection data recorded by the detection unit, wherein the detection unit has at least one camera, in particular, a 2D or 3D camera, and/or at least one movement sensor. By detecting the detection data with a camera and/or a movement sensor, the evaluation of the detection data can be performed reliably, since two different types of data can be detected and evaluated. For example, a video recorded by the camera needs only be evaluated to identify the type of action, during the times that movement is detected by the movement sensor. The two different types of detection data can be compared automatically so that errors in evaluating the detected data can be prevented. Preferably, the camera used is an optical camera. The optical camera takes images in series, which when evaluated provide information, whether a particular action has taken place. For example, a particular action may be identified when a specific pattern is recognized in the detection data. It is possible for example, for the operator or user to wear clothes of a particular color that is recognized easily by the detection unit or camera respectively. In such a case, the actions performed by the operator have to be actions belonging to the categories “patient preparation” or “patient release”.

According to a further embodiment of the invention, the step of identifying the type of the at least one action is based on a Deep Learning algorithm. The Deep Learning algorithm belongs to a class of optimization methods using artificial neural networks. The advantage of using such a Deep Learning algorithm is that by repeatedly detecting the same type of actions, the identifying of said action becomes more reliable each time it is detected.

According to a further embodiment of the invention, the at least one action is one of the following actions:
a patient enters the examination room,
a patient is placed on the patient table,
a device used for performing the examination, in particular a magnetic resonance coil, ear protection, and/or an emergency button, are placed on the patient,
establishing a vascular access to administer a medicine, in particular a contrast agent,
the patient is moved into an isocenter of the imaging apparatus,
the imaging apparatus is acquiring navigator images,
the imaging apparatus is acquiring diagnostic images
the patient is moved out of the isocenter,
the device used for performing the examination, in particular the magnetic resonance coil, ear protection and/or emergency button, are removed from the patient,
the patient gets up from the patient table,
the patient leaves the examination room,
maintenance staff for performing maintenance work is in the examination room and/or
nobody is present in the examination room.

The different types of actions that are detected belong to different types of categories. This means that the different types of actions can be aggregated to different categories, such as “patient registration”, “patient preparation”, “image acquisition” etc. The analysis can thus be focused on these categories that show which type of actions require most of the time and which type of actions require the least time of an examination. Such accurate analysis allows for an optimized utilization of the imaging apparatus, in particular it can allow for a parallelization of the work steps that are needed to be performed.

According to a further embodiment of the invention, the data entered into and/or recorded by the imaging apparatus, in particular acquisition times and/or registration data and/or registration times of a patient, are considered in order to perform a reliable analysis. This means different types of actions are detected by the detection unit and that the data acquired during the examination as such are analyzed and used to perform a proper analysis of the entire examination, in particular as a function of time. This has the particular advantage that a complete analysis can be performed automatically.

According to a further embodiment of the invention, the method includes the additional step of classifying the at least one identified action into different categories, wherein the different categories refer to generic terms such as registration, acquisition time, adjustment time, or maintenance time. The term “registration” means the registration of the patient by entering the name of the patient and/or the weight and/or the age and/or the already known anamnesis of the patient into a control unit of the imaging apparatus. The image acquisition time includes the time intervals for performing the medical examination as such. The adjustment time includes the time intervals for preparing the patient for the examination, for example, by placing coils, ear protection, and the like on or close to the patient. The maintenance time includes the time intervals for performing maintenance work on the imaging apparatus. By assigning the different types of detected actions to different categories, the analysis of utilization of the imaging apparatus can be performed accurately and in detail.

According to a further embodiment of the invention, the sequence of identified actions in one patient examination is analyzed with respect to the corresponding recorded times and time intervals of each identified action, and, in particular, the categories of the identified actions. Therewith, the time needed for each patient can be estimated reliably. This has the advantage that the daily utilization of the imaging apparatus can be planned such that the dead time of the imaging apparatus can be minimized.

According to a further embodiment of the invention, the analysis processor analyses a number of patient examinations and calculates an average time interval for each category of identified actions for several imaging modalities, wherein the average time intervals are compared with one another. By analyzing a number of patient examinations, the average time interval for each category can be calculated. This allows for analyzing which action or which type of action requires which amount of time on average.

According to a further embodiment of the invention, the recorded times and/or time intervals of one imaging apparatus or several imaging modalities are shown on a monitor to a user. This has the advantage that the user can effectively plan the utilization of the different modalities so that dead times of an apparatus can be prevented.

According to the inventive method, the different types of actions detected as a function of time can be assigned to different categories, which in turn allows for a detailed and reliable analysis of the utilization of the imaging apparatus, in particular in regard of dead times of the imaging apparatus or the like. Such a reliable analysis may be used to optimize the time schedule of the different work steps in order to optimize the utilization of the imaging apparatus. Furthermore, the detected data of a single apparatus as well as of a number of image modalities established in a facility can be detected and compared with one another. Moreover, detected data of several facilities may be compared with one another. In this latter case the facilities can be connected to a cloud based network, for example like “Teamplay”.

The inventive method offers an effective tool to obtain a statistically reliable analysis. For example, particular imaging modalities may be identified that work efficiently and other imaging modalities may be identified that work less efficiently. Such an analysis in turn allows for qualitative investigations of the factors why a particular imaging apparatus works efficiently or less efficiently. This factor can be improved or prevented in order to optimize the utilization of an imaging apparatus.

Furthermore, it is possible to provide the detected data in a clear format to a client operating several imaging modalities. The detected data can be supplied via “Teamplay” to the client. In this way the client may realize which imaging apparatus is operated efficiently and which one is operated less efficiently. The client is thus given the possibility to improve the efficiency of the client's imaging modalities.

The invention also encompasses a medical imaging apparatus having a first detection unit that detects first detection data of a first position of a patient table in the examination room and/or a second detection unit that detects second detection data of a second position anywhere else in the examination room. The apparatus also has an analysis processor that for analyzes the detection data, and the first and the second detection units communicate the detected data to the analysis processor. A control computer controls the detection unit and the analysis processor so as to perform the method as described above.

According to the inventive imaging apparatus, the work steps of preparing an examination, performing the same, and processing follow-up work steps are recorded close to the patient table and/or close to a position anywhere in the examination room by the detection unit. Since the different work steps are recorded continuously as a function of time, the different work steps are recorded accurately. This allows the analysis processor to analyze in detail the workflow of the imaging apparatus, such as a computer tomography apparatus, a magnetic resonance apparatus, or the like. The control computer controls the detection unit and the analysis processor so that the control computer controls the detection unit so as to supply the analyzing processor with the detected data. Furthermore, the control computer is configured to supply the analysis processor with data detected by the imaging apparatus during an examination. Since a first position is detected that indicates whether an action has taken place in the area of the patient table, and since at least a second position anywhere else in the examination room is detected that identifies whether an action has taken place in another area of the examination room, the detected data can be recorded accurately and completely, allowing an reliable analysis of the usage of the imaging apparatus. The actions in the other area can be, for example, detection of whether a coil needed for the examination is placed, monitoring an access to the examination room, or the like.

According to a further embodiment of the inventive imaging apparatus, the detection unit has at least one camera, in particular a 3D camera, for recording optical images, and/or at least one movement sensor for recording the at least one action, in particular a movement. Further examples of a movement sensor are, for example, a breathing sensor for monitoring the breathing of a patient, or by a sensor for monitoring an electrocardiogram for monitoring the cardiac activity of a patient. The detected data detected by the detection unit can be analyzed by using a Deep Learning algorithm. This means that the analysis can be performed automatically.

According to a further embodiment of the inventive imaging apparatus, the detection data of the first and the second detection units are analyzed individually. After analyzing the detected data of the first and the second detection units individually, the detection data are assigned to a particular category. In this way the detection data of one category that are acquired in different examinations can be compared with one another and/or averaged.

According to a further embodiment of the inventive imaging apparatus, the analysis processor is supplied with data entered into and/or recorded by the imaging apparatus for performing a reliable analysis. The data entered into the imaging apparatus includes the data that are aggregated under the category registration, for example the name, the weight, the age and/or the anamnesis of a patient. The data recorded by the imaging apparatus includes the data detected during an examination of the patient. The data recorded by the imaging apparatus can be magnetic resonance data, chromatography data, or the like. The data recorded can include times and durations of image acquisitions, etc.

In a further embodiment of the inventive imaging apparatus, the second position is at or close to the access to the imaging apparatus and/or the connection area of a magnetic resonance coil used for the examination. By detecting the second position, all actions or most of the actions taking place besides the patient table can be detected and can be analyzed. For example the coils used for a particular examination are usually plugged into the respective socket during patient preparation. Therefore the plugging and the unplugging of the corresponding coils are detectable as at least one action that is detected in the first or the second position. Therewith, the analysis of the utilization of an imaging apparatus can be improved.

The present invention also encompasses a non-transitory, computer-readable data storage medium encoded with programming instructions that, when the storage medium is loaded into a computer or computer system of a magnetic resonance apparatus, cause the computer or computer system to operate the magnetic resonance apparatus in order to implement any or all of the embodiments of the method according to the invention, as described above.

### BRIEF DESCRIPTION OF THE DRAWINGS

FIG. 1 schematically illustrates a magnetic resonance apparatus according to the invention.

FIG. 2 is a flowchart of the inventive method.

FIG. 3 shows an example of a time schedule for the utilization of the detection unit and the imaging apparatus during an examination of a patient.

### DESCRIPTION OF THE PREFERRED EMBODIMENTS

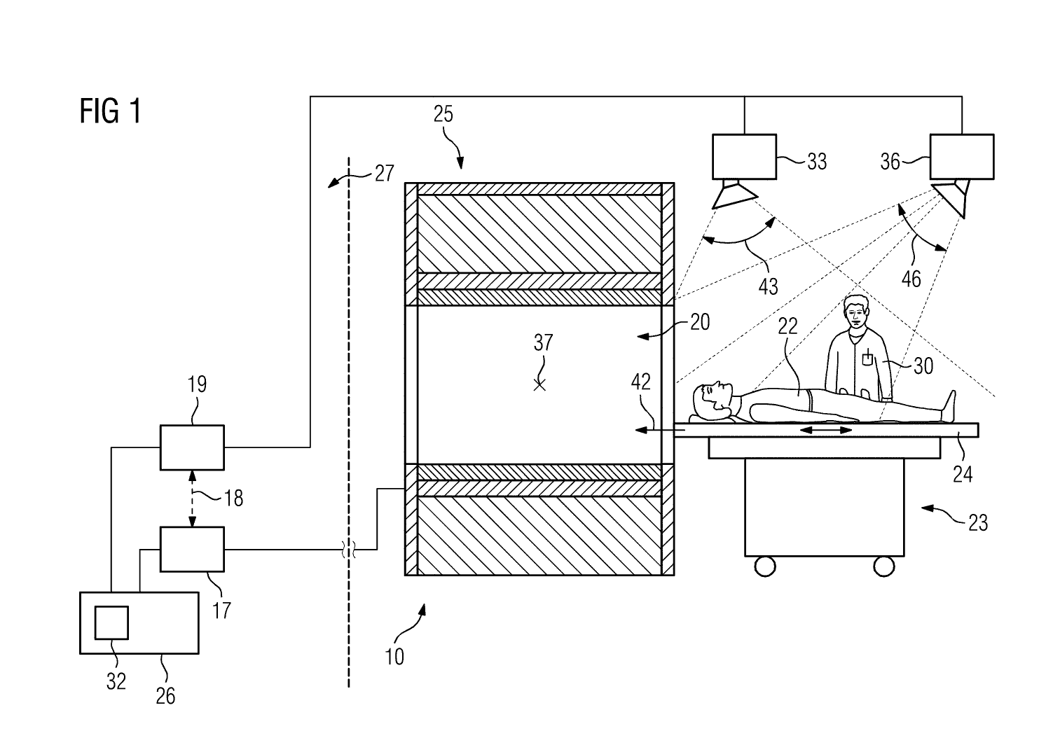

The FIG. 1 shows an imaging apparatus 10, for example, a magnetic resonance apparatus, a tomography apparatus, or the like. The scanner of the imaging apparatus 10 shown in FIG. 1 has an isocenter 37 into which a patient table 24 of a patient support 23 is movable. The patient table 24 of the patient support 23 on which a patient 22 is placed is movable along a direction 42 into the isocenter 37 of the imaging apparatus 10. The isocenter 37 is located in an acquisition area 20 of the scanner of the imaging apparatus 10. A user 30, in particular a physician, places a device used during the examination, for example, a magnetic resonance coil or ear protection, on the patient 22 before the patient is moved into the acquisition area 20. The imaging apparatus 10 and the patient table 24 are located in an examination room 25. The examination room 25 is recorded by a first detection unit 33 and/or a second detection unit 36. The first detection unit 33 is positioned such that it detects the actions performed close to the patient table 24. The second detection unit 36 detects a second position anywhere else in the examination room 25, in particular, an access to the examination room or an area for connecting an examination coil needed for the examination or the like.

The first detection unit 33 is pivotable through an angle range 43 and the second detection unit 36 is pivotable through an angle range 46. The first detection unit 33 and the second detection unit 36 each has a camera and/or a movement sensor for detecting whether an action has taken place. The first detection unit 33 and the second detection unit 36 are electronically connected to a control computer 19. The control computer 19 is connected to an analysis processor 26, so that the control computer 19 can supply the received detection data from the first and/or the second detection unit 33, 36 to the analysis processor 26. The analysis processor 26 is connected to an acquisition unit 17 or the imaging apparatus 10, which acquires the image acquisition data detected during an examination. The imaging apparatus 10 supplies the detected image acquisition data to the analyzing processor 26. The control computer 19 is connected to the acquisition unit 17 via the connection 18. The control computer 19 can control the acquisition unit 17 such that it allows the acquisition unit 17 to supply the acquisition data to the analysis processor 26 only when the first detection unit 33 detects that the patient table 24 is positioned, in particular, mainly within the acquisition area 20. The analysis processor 26 is provided with a monitor 32 at which the analysis of the detection data can be shown to a user 30 and which is positioned in a control room 27 that is adjacent to the examination room 25.

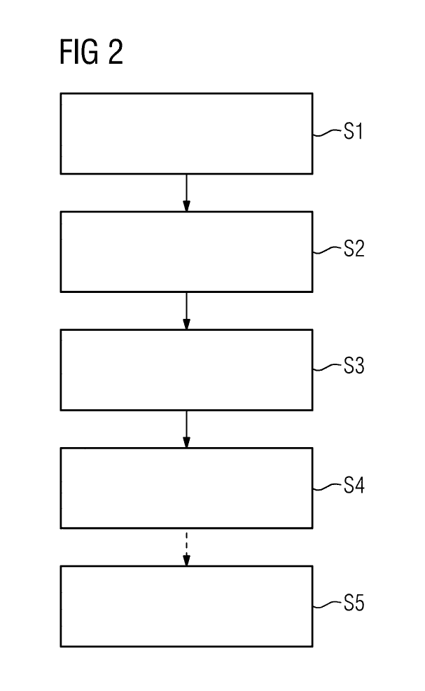

FIG. 2 shows a flowchart of the proposed method. In a first step S1 a first and/or a second detection data is recorded in, respectively, a first or second position in the examination room 25. In step S1, the first and the second detection data are recorded continuously as a function of time.

In a second step S2, the detection data are analyzed by the analysis processor 26. In the third step S3, it is identified whether at least one action has taken place. If an action has taken place, the time and/or the time interval in which the at least one action has taken place is evaluated. In the fourth step S4, the type of the at least one action that has taken place is identified and recorded. The identification of an action that has taken place and/or the type of action is analyzed using a Deep Learning algorithm.

In a fifth step S5, the analysis processor 26 is supplied with data, in particular, raw data that have been entered and/or recorded by the acquisition unit 17 of the imaging apparatus 10.

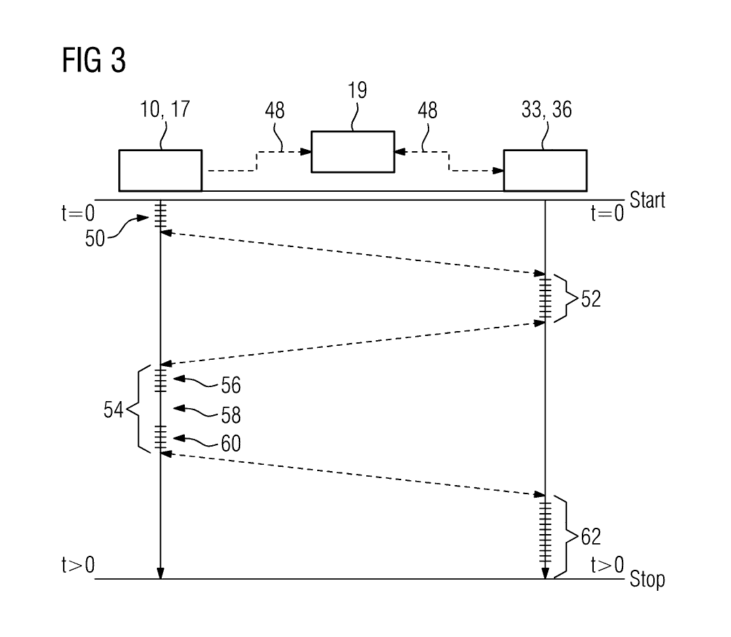

FIG. 3 shows an example of a time schedule of the utilization of the detection unit and the imaging apparatus during one examination of a patient 22. An examination starts at a time t=0 when a user 30 or an operator respectively starts entering patient information into the imaging apparatus 10. This step corresponds to the category “registration 50”. The same examination stops at a time t>0, when the patient leaves the examination room 25.

The registration 50 includes, for example, typing in the patient's name, the patient's age, the patient's sex, the patient's weight, the patient's height, or the like into the imaging apparatus 10. As long as the operator or user 30 enters the patient information during the registration 50 into the imaging apparatus 10, the detection units 33, 36 are expected to detect no action. Thus the detection units 33, 36 are in a “stand-by mode” corresponding to a detection of a dead time interval. The patient information are typed into the imaging apparatus 10 and are automatically supplied to the control computer 19 through a connection 48.

After the registration 50 is performed the patient 22 is prepared for the examination. The actions performed for the “patient preparation 52” are detected by the detection units 33, 36 and may comprise for example the actions “the patient enters the examination room 25”, “placing the patient 22 on the patient table 24”, “placing a magnetic coil used for the examination on the patient 22” or “plugging said coil into a predetermined position on the patient table 24”, “establishing a vascular access to administer a medicine”, or the like. During the step of patient preparation 52 the detection units 33, 36 are in an active mode, while the imaging apparatus 10 is preferably in an inactive mode, i.e. a stand-by mode. The inactive mode corresponds to a state, in which the imaging apparatus 10 acquires or receives no data. The detection data detected by the detection units 33, 36 are automatically supplied to the control computer 19 through the connection 48. The supply of data from the imaging apparatus 10 or from the detection units 33, 36 is indicated by reference numeral 48.

After the step “patient preparation 52” is performed a step of “image acquisition 54” follows, in which the imaging apparatus 10 is in an active mode. During the step of image acquisition 56 the following actions may be detected by the imaging apparatus 10. At the beginning of the step of image acquisition 54, navigator images may be detected in a sub-step 56. After the navigator images are detected in sub-step 56, these navigator images may be evaluated by the operator in a further sub-step 58. During evaluation of the navigator images in sub-step 58, the imaging apparatus is in an inactive mode, i.e. a dead time of the imaging apparatus is detected. After the evaluation of the navigator images in sub-step 58 a further sub-step 60 may follow, in which diagnostic images are acquired. The acquisition of the diagnostic images correspond to the proper examination of the patient 22. The sub-steps 56 and 60 are comprised in the category of “image acquisition 54”. The step 58 may be in a category “dead time of the imaging apparatus”. While the step 54 is performed, the detection units 33, 36 usually detect no action. Thus the detection units 33, 36 are in an inactive mode, like a stand-by mode.

After the image acquisition 54 is finished, the patient 22 is removed from the imaging apparatus 10, by moving out the patient table 24 from the imaging apparatus 10. In a step of a “patient release 62” the detections units 33, 36 are again in an active mode for detecting actions like, for example, releasing the patient 22 from the vascular access or the like, the patient 22 gets up from the patient table 24, the patient 22 leaves the examination room 25. After the patient 22 has left the examination room 25 the examination is completed.

In summary, this means a particular examination starts with step 50, when the patient is registered and stops when the patient leave the examination room 25 in step 62. When the steps 50 to 62 are performed either the imaging apparatus 10 is active, while the detection units 33, 36 are usually inactive, or the detection units 33, 36 are active, while the imaging apparatus 10 is usually inactive.

However, it is possible to perform the steps 50 and 52 in parallel. In this case a first operator may enter the patient information, and a second operator may prepare the patient. Since however the detected data are supplied automatically to the control computer 19 as a function of time, all the detected actions may be graded as a function of time and as a function of a particular category.

The inventive method for detecting and analyzing the workflow performed with an imaging apparatus provides a tool with which the utilization of the imaging apparatus can be evaluated and improved.

Although modifications and changes may be suggested by those skilled in the art, it is the intention of the Applicant to embody within the patent warranted hereon all changes and modifications as reasonably and properly come within the scope of the Applicant's contribution to the art.

1. A method for detecting and analyzing a workflow with which examination of a patient is executed by a data acquisition scanner of a medical imaging apparatus, said data acquisition scanner being situated in an examination room and comprising a patient table adapted to receive the patient thereon, said workflow comprising a plurality of work steps involving respective occurrences of different types of actions, said method comprising:
operating a detection unit in order to record first detection data and second detection data continuously over time, said first detection data representing a first position on or adjacent to the patient table in the examination room, and said second detection data representing a second position that is anywhere else in the examination room other than said first position;
providing said first and second detection data to an analysis processor and, in said analysis processor, analyzing said first and second detection data in order to identify whether at least one of said actions has occurred in the examination room and, when an occurrence of at least one of said actions is identified, recording a time or time interval in which said at least one action occurred, and identifying and recording a type of said at least one action; and
generating an archive record representing all identified actions and times and time intervals and types of all actions that are identified as occurring in said first and second detection data, and making said archive record available from the analysis processor in an electronic form that allows review and evaluation of said archive record.

2. A method as claimed in claim 1 comprising identifying whether said at least one action has occurred based on detection of a movement represented in either said first detection data or said second detection data.

3. A method as claimed in claim 1 comprising recording said first and second detection data using, as said detection unit, at least one of a 2D camera, a 3D camera, and a movement sensor.

4. A method as claimed in claim 1 comprising identifying said type of said at least one action by executing a deep learning algorithm in said analysis processor.

5. A method as claimed in claim 1 wherein said at least one action is selected from the group consisting of the patient entering the examination room, the patient being placed on the patient table, an accessory for performing said examination being placed on the patient, establishing vascular access to the patient in order to administer a fluid involved in said examination, moving the patient into an isocenter of the data acquisition scanner, operating the data acquisition scanner in order to acquire navigator images, operating the data acquisition scanner in order to acquire diagnostic images, moving the patient out of the isocenter of the data acquisition scanner, removing said accessory from the patient, the patient moving from the patient table, the patient exiting the examination room, performance of maintenance work in the examination room, and an absence of personnel and the patient in the examination room.

6. A method as claimed in claim 1 comprising entering examination-specific data into a computer of the medical imaging apparatus prior to beginning said examination, and analyzing said first and second detection data in said analysis processor together with said data entered into said computer of said medical imaging apparatus, said data entered into said computer of said medical imaging apparatus comprising at least one of image acquisition times and patient registration data.

7. A method as claimed in claim 1 comprising classifying said at least one action into a category selected from the group consisting of patient registration time, medical data acquisition time, adjustment time, and maintenance time.

8. A method as claimed in claim 7 comprising analyzing said archive record with respect to said times and time intervals of said at least one action that occur respectively in said categories.

9. A method as claimed in claim 1 comprising producing said archive record for each of a plurality of examinations of multiple patients, and evaluating said archive records by calculating an average time interval for each type of action identified in the respective archive records, and comparing the respective average time intervals for the respective examinations with each other.

10. A method as claimed in claim 1 comprising presenting the times or time intervals in said archive record at a monitor so as to make said times to time intervals available to an operator of the medical imaging apparatus.

11. A medical imaging apparatus comprising:
a data acquisition scanner situated in an examination room and comprising a patient table adapted to receive a patient thereon;
a computer configured to operate the data acquisition scanner to execute an examination of the patient, said workflow comprising a plurality of work steps involving respective occurrences of different types of actions;
a detection unit that records first detection data and second detection data continuously over time, said first detection data representing a first position on or adjacent to the patient table in the examination room, and said second detection data representing a second position that is anywhere else in the examination room other than said first position;
an analysis processor provided with said first and second detection data, said analysis processor being configured to analyze said first and second detection data in order to identify whether at least one of said actions has occurred in the examination room and, when an occurrence of at least one of said actions is identified, to record a time or time interval in which said at least one action occurred, and to identify and record a type of said at least one action; and
said analysis processor being configured to generate an archive record representing all identified actions and times and time intervals and types of all actions that are identified as occurring in said first and second detection data, and to make said archive record available from the analysis processor in an electronic form that allows review and evaluation of said archive record.

12. A medical imaging apparatus as claimed in claim 11 wherein said detection unit is a motion detector.

13. A medical imaging apparatus as claimed in claim 12 wherein said motion detector is selected from the group consisting of a 2D camera, a 3D camera, and a movement sensor.

14. A non-transitory, computer-readable data storage medium encoded with programming instructions for detecting and analyzing a workflow with which examination of a patient is executed by a data acquisition scanner of a medical imaging apparatus, said data acquisition scanner being situated in an examination room and comprising a patient table adapted to receive the patient thereon, said workflow comprising a plurality of work steps involving respective occurrences of different types of actions, said storage medium being loaded into a computer system of a medical imaging apparatus and said programming instructions causing said computer system to:
operate a detection unit in order to record first detection data and second detection data continuously over time, said first detection data representing a first position on or adjacent to the patient table in the examination room, and said second detection data representing a second position that is anywhere else in the examination room other than said first position;
analyze said first and second detection data in order to identify whether at least one of said actions has occurred in the examination room and, when an occurrence of at least one of said actions is identified, record a time or time interval in which said at least one action occurred, and identify and recording a type of said at least one action; and
generate an archive record representing all identified actions and times and time intervals and types of all actions that are identified as occurring in said first and second detection data, and make said archive record available from the computer system in an electronic form that allows review and evaluation of said archive record.
